(19) United States
(12) Patent Application Publication
(10) Pub. No.: US 2023/0197234 A1
(43) Pub. Date: Jun. 22, 2023

(54) CONTAINER VAULT SYSTEM

(71) Applicant: Evernorth Strategic Development, Inc., St. Louis, MO (US)

(72) Inventors: Moses Vaughan, Nutley, NJ (US); Christopher M. Myers, Dublin, OH (US); Bill Loi Pham, Highlands Ranch, CO (US); Harinath Kasina, Manalapan, NJ (US)

(21) Appl. No.: 17/555,704

(22) Filed: Dec. 20, 2021

(51) Int. Cl. G16H 20/13; G16H 70/40; G16H 80/00; G06F 21/32

(52) U.S. Cl. CPC G16H 20/13; G16H 70/40; G16H 80/00; G06F 21/32; G06F 2221/2141

(57) ABSTRACT

A container vault system includes a plurality of container vaults in communication with a server via a communication network. The server is configured to store package data for each of a plurality of medication packages. Each of the container vaults comprises at least one container configured to hold a medication package, as well as a control system configured to (i) receive the package data for the medication package from the server, wherein the package data identifies at least one authorized user permitted to retrieve the medication package and (ii) control access to the container so that the authorized user identified in the package data is permitted to retrieve the medication package from the container.

### CROSS-REFERENCE TO RELATED APPLICATIONS

Not applicable.

### STATEMENT REGARDING FEDERALLY SPONSORED RESEARCH OR DEVELOPMENT

Not applicable.

### STATEMENT REGARDING JOINT RESEARCH AGREEMENT

Not applicable.

### BACKGROUND OF THE INVENTION

Pharmaceutical fulfillment systems fill a large number of prescription orders with a variety of medications. Generally, a pharmacist fills each prescription order by placing a specific quantity of medication in a container. A logistics company then transports each container in a shipping package to the consumer who placed the prescription order. While some medications are unaffected by temperature and can be delivered to consumers in standard shipping packages, others must be stored within a specific temperature range. These temperature-sensitive medications are typically delivered to consumers in temperature-controlled shipping packages, such as various types of coolers.

There are a number of problems associated with conventional methods for transporting medication packages to consumers. For example, both standard and temperature-controlled shipping packages do not provide any meaningful security features to guard against theft or receipt of the medication packages by impersonating identities. This can result in fraud and potential prescription drug abuse by unauthorized users. Also, while coolers and other temperature-controlled shipping packages provide a certain level of temperature protection, they may not be sufficient to protect high end specialty drugs that have specific environmental storage requirements. This issue is particularly problematic when shipping packages sit outside in extreme weather conditions. Thus, there remains a need in the art for a technological solution that offers features, functionality or other advantages not provided by existing packaging solutions.

### BRIEF SUMMARY OF THE INVENTION

The present invention is directed to a container vault system for controlling access to medication packages and preferably storing the medication packages under desired environmental conditions. The system includes a central server in communication with a plurality of container vaults via a communication network. In some embodiments, the container vaults are locker-type facilities located at different geographic locations, such as within pharmacies or other retail stores. Each container vault includes a plurality of containers each of which holds a medication package transported from a pharmaceutical fulfillment system, wherein a consumer or other authorized user retrieves the medication package from the container vault. In some embodiments, the container vault comprises a single standalone enclosure that holds a medication package transported from a pharmaceutical fulfillment system directly to a consumer.

In some embodiments, the container vault includes a control system that receives package data for each medication package from the central server, wherein the package data identifies at least one authorized user permitted to retrieve the medication package. The control system controls access to the container so that only the authorized user identified in the package data is permitted to retrieve the medication package from the container. In one aspect, the control system controls access to the container by obtaining access credentials via an input device (e.g., a card reader, a barcode scanner, a touchscreen display, a keypad, or a biometric reader) and determining if the access credentials are associated with the authorized user. If the determination is affirmative, the control system unlocks the vault to enable access to the container and medication package therein.

In some embodiments, the container vault includes a control system that receives package data for each medication package from the central server, wherein the package data identifies at least one environmental property setting to be used for storage of the medication package. The environmental property may comprise, for example, a temperature level, humidity level, light level, pressure level, or ventilation level within the container. The control system adjusts the environmental property within the container to achieve the environmental property setting.

In some embodiments, the central server defines a group containing one or more of the container vaults, and analyzes at least a portion of the package data for the medication packages retrieved from those container vaults to identify a group characteristic. The group characteristic may comprise, for example, a medical condition associated with the group or a medical supply needed for the group. In one aspect, the central server tracks the group characteristic either within the group or in relation to one or more additional groups. In another aspect, the central server transmits a notification of the group characteristic to one or more computing devices of one or more health care providers. The notification of the group characteristic may include, for example, educational materials for a medical condition associated with the group or a medical supply list for the group.

Various embodiments of the present invention are described in detail below, or will be apparent to one skilled in the art based on the disclosure provided herein, or may be learned from the practice of the invention. It should be understood that the above brief summary of the invention is not intended to identify key features or essential components of the embodiments of the present invention, nor is it intended to be used as an aid in determining the scope of the claimed subject matter as set forth below.

### BRIEF DESCRIPTION OF THE DRAWINGS

A detailed description of various exemplary embodiments of the present invention is provided below with reference to the following drawings, in which:

FIG. 1 is a network diagram of a container vault system in accordance with one embodiment of the invention;

FIG. 2 is a block diagram of the central server shown in FIG. 1;

FIG. 3 is a front perspective view of an exemplary container vault that may be used as one of the container vaults shown in FIG. 1;

FIG. 4 is a front perspective view of the container vault shown in FIG. 3 with the top doors and containers removed to show the magnetic induction rails on the floor of each compartment of the vault housing;

FIG. 5 is a front perspective view of an exemplary container that may be placed in a compartment of the vault housing shown in FIGS. 3 and 4;

FIG. 6 is a block diagram of the flooring section of the container shown in FIG. 5;

FIG. 7 is a block diagram of the electronic circuit incorporated into the flooring section shown in FIG. 6;

FIG. 8 is a block diagram of the control system of the container vault shown in FIGS. 3 and 4;

FIG. 9 is a process flow diagram of an exemplary method of managing the order and transport of a medication package as performed by the central server shown in FIG. 1;

FIGS. 10A and 10B are process flow diagrams of an exemplary method of managing the intake, storage and retrieval of a medication package as performed by the container vault shown in FIG. 1;

FIG. 11 is a process flow diagram of an exemplary method of tracking the group characteristics of various container vault groups as performed by the central server shown in FIG. 1;

FIG. 12 depicts a circular geofence associated with one of the container vaults shown in FIG. 1;

FIG. 13 depicts a polygon geofence associated with one of the container vaults shown in FIG. 1;

FIG. 14 depicts a group of the container vaults shown in FIG. 1 with their corresponding circular geofences;

FIG. 15 is a front perspective view of a transportable container that may be placed in a compartment of the vault housing shown in FIGS. 3 and 4; and

FIG. 16 is a front perspective view of an exemplary standalone container vault that may be used as one of the container vaults shown in FIG. 1.

### DETAILED DESCRIPTION OF EXEMPLARY EMBODIMENTS

The present disclosure is directed to a container vault system for controlling access to medication packages and preferably storing the medication packages under desired environmental conditions. While the invention will be described in detail below with reference to various exemplary embodiments, it should be understood that the invention is not limited to the specific configurations or methods of any of these embodiments. In addition, although the exemplary embodiments are described as embodying several different inventive features, those skilled in the art will appreciate that any one of these features could be implemented without the others in accordance with the invention.

In the present disclosure, references to “one embodiment,” “an embodiment,” “an exemplary embodiment,” or “embodiments” mean that the feature or features being described are included in at least one embodiment of the invention. Separate references to “one embodiment,” “an embodiment,” “an exemplary embodiment,” or “embodiments” in this disclosure do not necessarily refer to the same embodiment and are also not mutually exclusive unless so stated and/or except as will be readily apparent to one skilled in the art from the description. For example, a feature, structure, function, etc. described in one embodiment may also be included in other embodiments, but is not necessarily included. Thus, the present invention can include a variety of combinations and/or integrations of the embodiments described herein.

### System Configuration

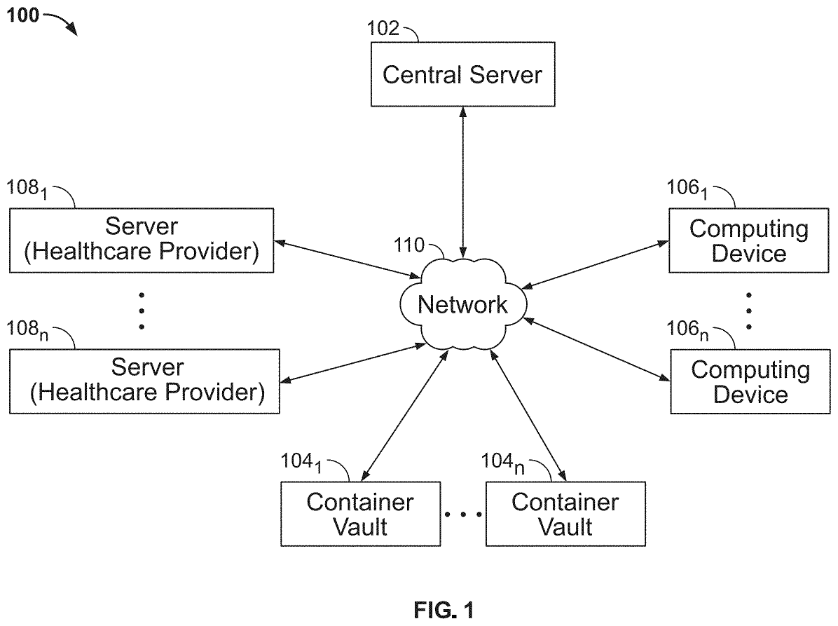

Referring to FIG. 1, a container vault system in accordance with one embodiment of the present invention is shown generally as reference number 100. System 100 includes a central server 102 that communicates with a plurality of network elements—i.e., a plurality of container vaults 104₁-104ₙ, a plurality of computing devices 106₁-106ₙ, and a plurality of healthcare provider servers 108₁-108ₙ—via a communication network 110.

Network 110 may comprise any network or combination of networks capable of facilitating the exchange of data among the network elements of system 100. In some embodiments, network 110 enables communication in accordance with one or more cellular standards, such as the Long-Term Evolution (LTE) standard, the Universal Mobile Telecommunications System (UMTS) standard, and the like. In other embodiments, network 110 enables communication in accordance with the IEEE 802.3 protocol (e.g., Ethernet) and/or the IEEE 802.11 protocol (e.g., Wi-Fi). Of course, other types of networks may also be used within the scope of the present invention.

### Central Server

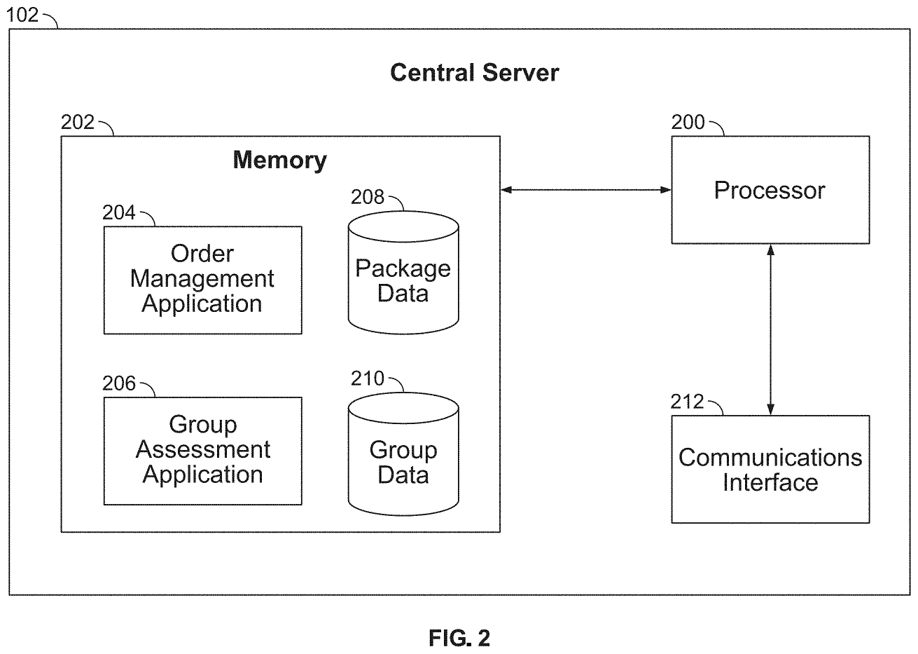

FIG. 2 is a block diagram illustrating an exemplary embodiment of central server 102 that may be used within system 100. Central server 102 includes a processor 200 that is operatively connected to various hardware and software components, which together enable central server 102 to perform the methods described herein. Processor 200 may comprise a multi-core processor, multiple processors, or some other type of processor, depending on the particular implementation. Processor 200 is operatively connected to a memory 202, which may be fixed or removable. Memory 202 may include any suitable combination of volatile memory (e.g., random-access memory (RAM)) and non-volatile memory (e.g. read-only memory (ROM), electrically erasable programmable read-only memory (EEPROM), or flash memory). Memory 202 stores one or more software programs or applications, such as an order management application 204 and a container group assessment application 206, as well as one or more databases, such as a medication package database 208 and a container group database 210, as described below.

Order management application 204 includes one or more software modules each of which comprises instructions that, when executed by processor 200, cause processor 200 to perform certain methods described herein relating to the receipt of orders, creation of package data for each medication package, and transport of medication packages to container vaults 104₁-104ₙ (described below with reference to FIG. 9). Medication package database 208 contains package data for the medication packages transported to container vaults 104₁-104ₙ. The package data for each medication package may be stored in association with a globally unique identifier (GUID) for the container vault compartment in which the medication package is stored while waiting for retrieval by a consumer or other authorized user. In this embodiment, the package data generally includes (1) configuration data that identifies at least one environmental property setting to be used for storage of the medication package and (2) user access data that identifies at least one authorized user permitted to retrieve the medication package.

For example, the package data may include any combination of the following information: the destination container vault; the physical location of the container vault; the desired environmental properties for storage of the medication package (e.g., temperature level, humidity level, light level, pressure level, or ventilation level); the sender of the medication package (e.g., a pharmaceutical fulfillment system); a list of transporters for the medication package (e.g., a logistics company); the intended recipient of the medication package (e.g., a consumer/patient); a list of collectors authorized to retrieve the medication package (e.g., a family member, caretaker, or other authorized user); a medication package value; a list of medication package category tags; the time that the mediation package is placed in the container vault; the time that the medication package is retrieved from the container vault; and a status of the medication package. Of course, other types of package data may also be stored in accordance with the present invention.

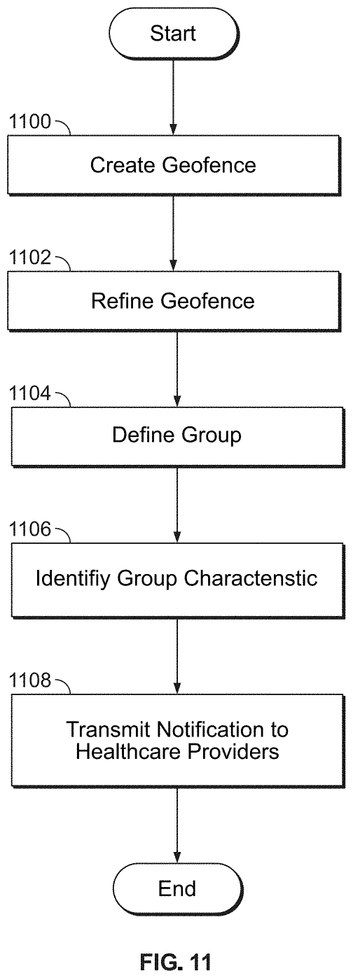

Container group assessment application 206 includes one or more software modules each of which comprises instructions that, when executed by processor 200, cause processor 200 to perform certain methods described herein relating to the assessment and tracking of group characteristics for various groups of container vaults 104₁-104ₙ (described below with reference to FIG. 11). Container group database 210 contains group data that generally includes a group characteristic for each container group. The group characteristic may comprise, for example, a medical condition associated with the container group or a medical supply needed for the container group. Of course, other types of group characteristics may also be stored in accordance with the present invention.

Central server 102 further includes a communications interface 210 that is operatively connected to processor 200. Communications interface 210 may be any interface that enables communication between central server 102 and other network elements (including container vaults 104₁-104ₙ, computing devices 106₁-106ₙ, and healthcare provide servers 108₁-108ₙ) via network 110. In some embodiments, the communications interface 210 comprises a network interface card (NIC), an integrated network interface, and/or any other interfaces for connecting central server 102 to other network elements and/or communication networks. Such connections can include a wired connection or a wireless connection.

In the embodiment shown in FIG. 1, central server 102 is used to store medication package management application 204, container group assessment application 206, medication package database 208, and container group database 210. Of course, other embodiments may include additional servers for performing some of these methods, which may be co-located in the same geographic location or located in different geographic locations and connected to each other via network 110. For example, all or a portion of the various modules of medication package management application 204 and/or container group assessment application 206 could be stored on one or more application servers that may be accessed via network 110. Further, all or a portion of the data contained in medication package database 208 and/or container group database 210 could be stored on one or more database servers that may be accessed via network 110. Thus, the system may be implemented with any number and combination of servers that are either co-located or geographically dispersed.

### Container Vaults

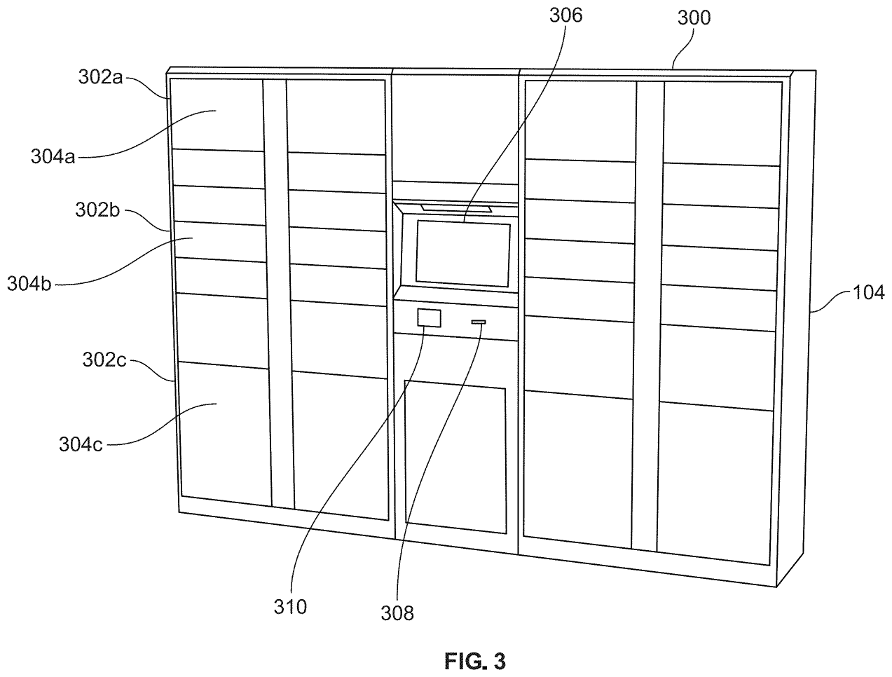

FIG. 3 illustrates an exemplary embodiment of a container vault 104 that may be used as one of container vaults 104₁-104ₙ shown in FIG. 1. Container vault 104 comprises a locker-type facility that may be located within a pharmacy or other retail store. Container vault 104 includes a vault housing 300 that defines a plurality of vault compartments, including vault compartment 302a, vault compartment 302b, and vault compartment 302c. In this embodiment, container vault 104 includes twenty-nine vault compartments—i.e., sixteen small-sized vault compartment (such as vault compartment 302b), eight medium-sized vault compartments (such as vault compartment 302a), and five large-sized vault compartments (such as vault compartment 302c). Of course, a container vault may include any number and size of vault compartments in accordance with the present invention. Each of the vault compartments is assigned a globally unique identifier (GUID) that may be associated with package data for a medication package assigned to that vault compartment, as described below.

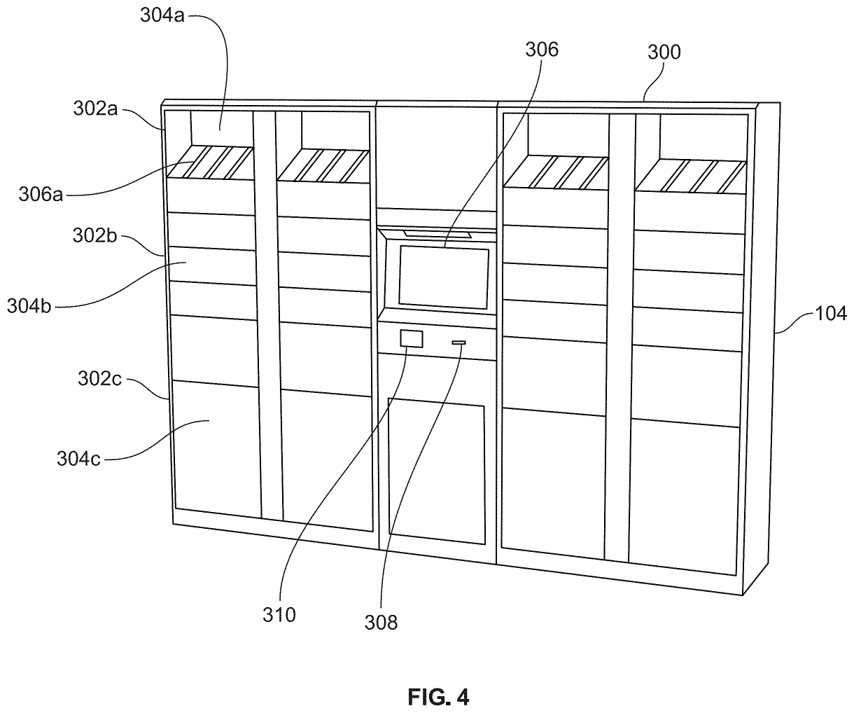

Each of the vault compartments is configured to house a removable container behind a locked door, such as doors 304a, 304b, 304c of vault compartments 302a, 302b, 302c, respectively. FIG. 4 shows container vault 104 without the doors on the top row of vault compartments (and without placement of the containers therein) to illustrate the interior structure of the vault compartments. As can be seen, the floor of each of the vault compartments includes magnetic induction rails, such as magnetic induction rails 306a of container vault 302a. It will be seen that the magnetic induction rails are used to wirelessly charge a rechargeable battery of the container housed in the vault compartment.

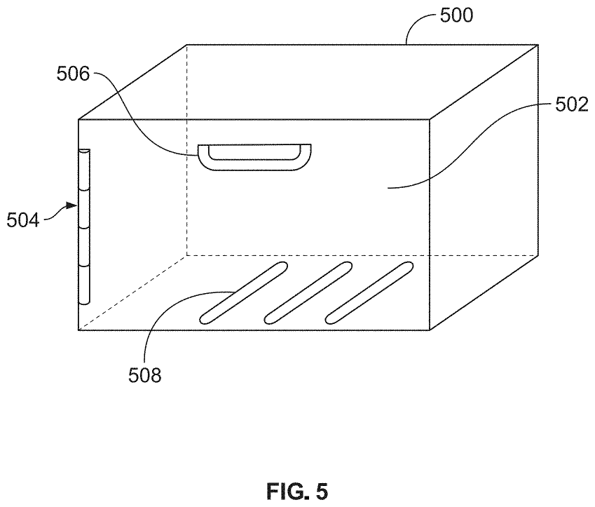

FIG. 5 illustrates an exemplary embodiment of a container 500 that may be housed within one of the vault compartments of container vault 104. As can be seen, container 500 comprises a generally box-like structure that is configured to hold a medication package transported from a pharmaceutical fulfillment system. Container 500 includes a door 502 that may be pivotally moved about a hinge 504 between an open position and a closed position. A handle 506 is provided on the exterior of door 502 for use in sliding container 500 out of its vault compartment. The bottom of container 500 includes charging contacts 508 that cooperate with the magnetic induction rails of the vault compartment (see FIG. 4) to wirelessly charge a rechargeable battery of container 500, as discussed below.

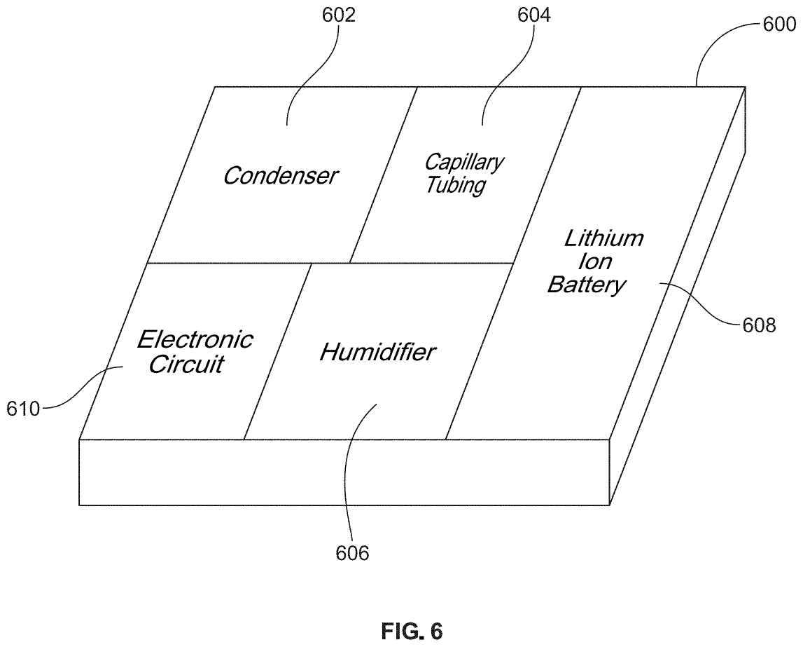

As shown in FIG. 6, container 500 includes a flooring section 600 that is positioned over charging contacts 508 on the bottom of container 500 (note that flooring section 600 is not shown in FIG. 5 to better illustrate charging contacts 508). Flooring section 600 includes one or more components that are configured to change an environmental property (e.g., temperature level, humidity level, light level, pressure level, and/or ventilation level) within container 500. In this embodiment, flooring section 600 includes the following components: a condenser 602 with evaporator coils that extract heat from the interior of container 500 to the outside of container vault 104 so as decrease the temperature within container 500; capillary tubing 604 that meters the refrigerant in the evaporator coils so as to adjust the temperature within container 500; and a humidifier unit 606 configured to adjust the humidity within container 500. Of course, flooring section 600 may include other components that are configured to change different environmental properties in accordance with the present invention.

Flooring section 600 also includes a rechargeable battery 608 that is configured to provide power to the various components of flooring section 600. In this embodiment, rechargeable battery 608 comprises a lithium-ion rechargeable battery with contacts positioned to contact charging contacts 508 on the bottom of container 500. As discussed above, charging contacts 508 cooperate with the magnetic induction rails of the vault compartment (see FIG. 4) to wirelessly charge rechargeable battery 608.

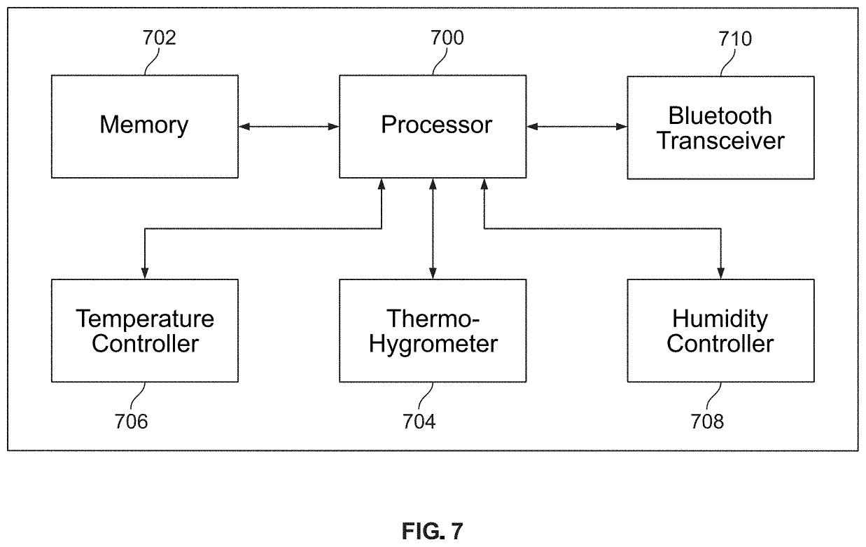

Flooring section 600 also includes an electronic circuit 610. FIG. 7 is a block diagram illustrating an exemplary embodiment of electronic circuit 610, which includes an onboard processor 700 that is operatively connected to a memory 702 and the following components: a thermo-hygrometer 704 configured to measure the temperature and humidity within container 500; a temperature controller 706 configured to control condenser 602 and capillary tubing 604 so as to adjust the temperature level within container 500; a humidity controller 708 configured to control humidifier unit 606 so as to adjust the humidity level within container 500; and a Bluetooth transceiver 710 configured to enable communication with the control system of container vault 104 in accordance with the IEEE 802.15.1 protocol. Of course, other types of communication interfaces may also be used in accordance with the present invention.

Memory 702 stores firmware execution code instructions that, when executed by processor 700, cause processor 700 to perform the steps required to maintain a desired temperature level and a desired humidity level within container 500. Specifically, processor 700 receives a temperature setting and a humidity setting to be used for storage of a medication package in container 500. In this embodiment, the settings are received from the control system of container vault 104 via Bluetooth transceiver 710, as described in greater detail below. Processor 700 receives periodic temperature and humidity measurements from thermo-hygrometer 704. If a temperature measurement is not equal to the received temperature setting, then processor 700 sends instructions to temperature controller 706 to adjust the temperature level within container 500 via control of condenser 602 and capillary tubing 604. Similarly, if a humidity measurement is not equal to the received humidity setting, then processor 700 sends instructions to humidity controller 708 to adjust the humidity level within container 500 via control of humidifier unit 606.

Referring back to FIG. 3, container vault 104 also includes one or more input devices that enable an authorized user to input the access credentials required to retrieve a medication package from one of the vault compartments. An authorized user may be the intended recipient of the medication package (e.g., a patient/consumer) or an individual who the intended recipient has authorized to retrieve the medication package (e.g., a family member or caretaker), as designated on a list of collectors. In some embodiments, the intended recipient is able to update the list of collectors via a user profile maintained on central server 102 or another server managed by the pharmaceutical fulfillment system.

In this embodiment, the input devices comprise a touchscreen display 306, a card reader 308, and a barcode scanner 310. Touchscreen display 306 is configured to provide a graphical user interface that enables a user to enter a personal identification number (PIN), e.g., a randomized PIN that is sent to the intended recipient via email or text message. Card reader 308 is configured to read a medical/insurance card, and barcode scanner 310 is configured to scan a barcode provided on a government issued identification card (e.g., a driver's license) or any other type of identification card with a barcode. Of course, other types of input devices may also be used in accordance with the invention, such as a physical keypad or biometric reader.

In some embodiments, an authorized user may use any one of the input devices to input the access credentials required to retrieve a medication package from one of the vault compartments. In other embodiments, a secondary factor of security is required, e.g., an individual must possess a card that is read by either card reader 308 or barcode scanner 310 and enter a PIN into touchscreen display 306. Of course, other authentication methods may also be used in accordance with the present invention.

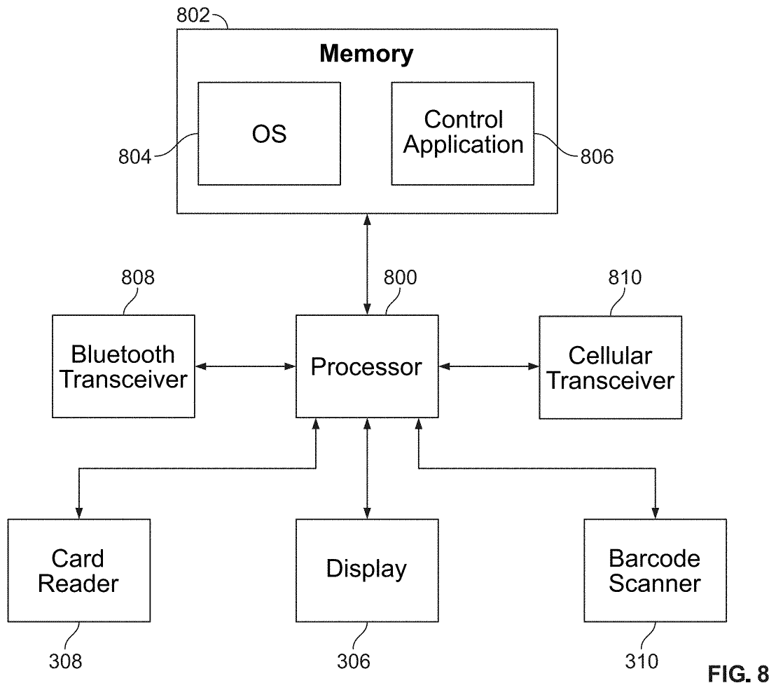

Container vault 102 further includes a control system that includes the input devices described above. FIG. 8 is a block diagram illustrating an exemplary embodiment of the control system that may be used within container vault 102. The control system includes a processor 800 that may comprise a central processing unit (CPU), a multi-core processor, multiple processors, or some other type of processor, depending on the particular implementation. Processor 800 is operatively connected to memory 802, which may comprise any suitable combination of volatile memory (e.g., random-access memory (RAM)) and non-volatile memory (e.g. read-only memory (ROM), electrically erasable programmable read-only memory (EEPROM), or flash memory). Memory 802 stores an operating system 804 and a control application 806.

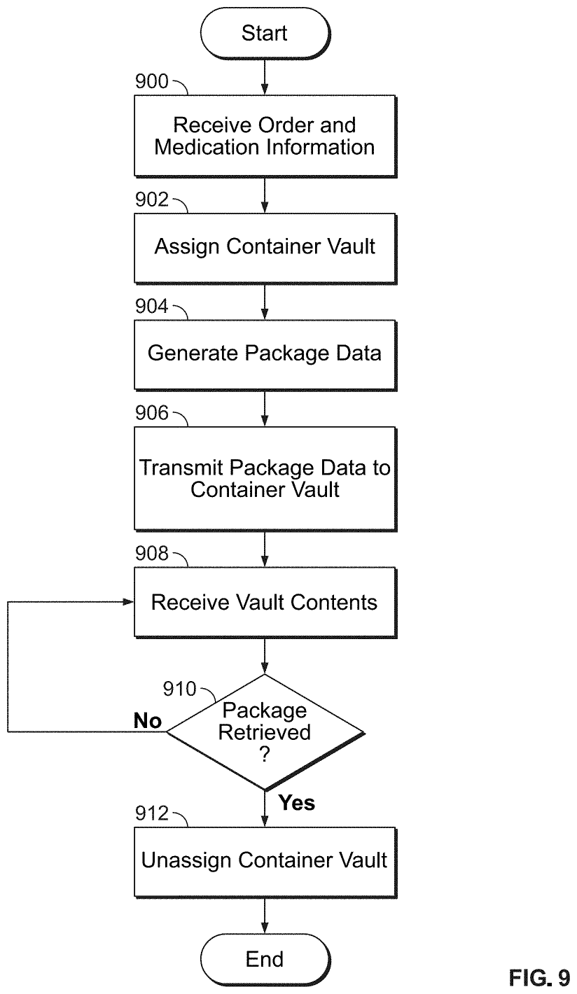

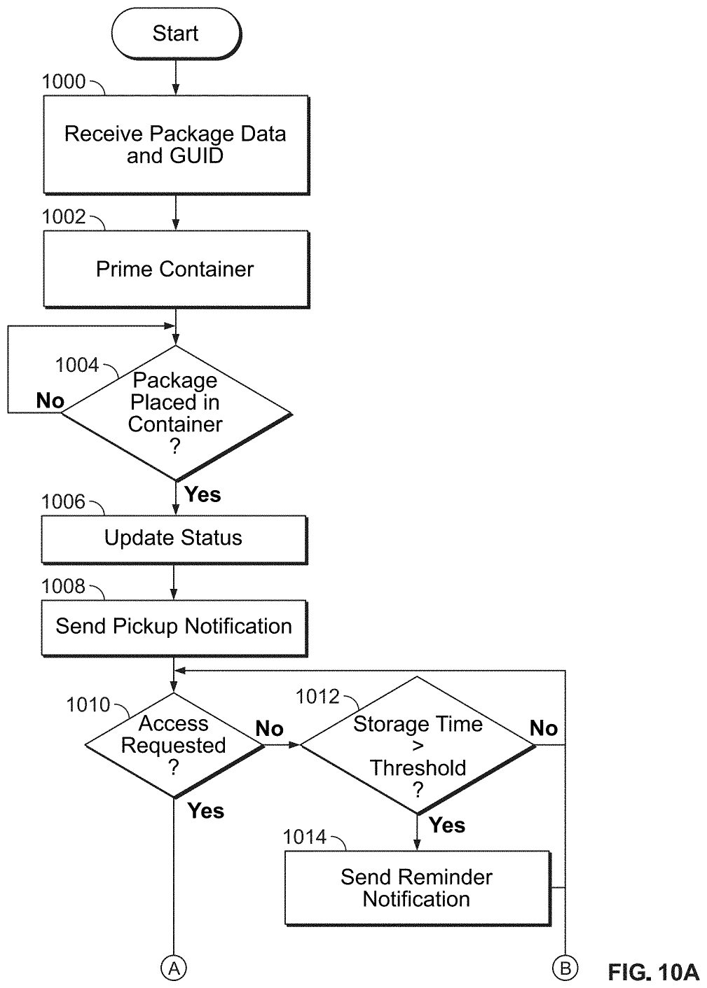

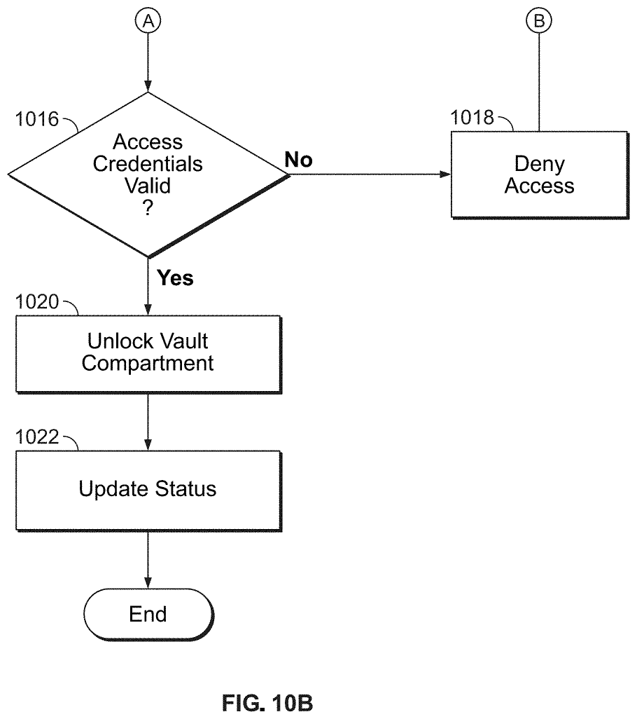

Control application 806 refers generally to an application that enables communication with central server 102 and each container of container vault 102. Control application 806 comprises instructions that, when executed by processor 800, cause processor 800 and the various hardware components of the control system to (1) control access to each container so that only authorized users are permitted to retrieve the medication package from the container and (2) control the environmental components within each container so as to achieve desired environmental property settings for storage of a medication package within the container (as described below in connection with FIGS. 10A and 10B).

Processor 800 is also operatively connected to a Bluetooth transceiver 808 that enables short-range communication with each of the containers of container vault 102 in accordance with the IEEE 802.15.1 protocol. Of course, other types of short-range communication interfaces may also be used in accordance with the present invention.

Processor 800 is further operatively connected to cellular transceiver 810 that enables long-range communication with central server 102 and computing devices 106₁-106ₙ in accordance with a cellular protocol, such as those that operate using the Long-Term Evolution (LTE) standard, the Universal Mobile Telecommunications System (UMTS) standard, and the like. Alternatively, in some embodiments, processor 800 connects to a local network through a wireless connection (e.g., Wi-Fi) or a hard-wired connection (e.g., Ethernet), which in turn enables communication with central server 102 and computing devices 106₁-106ₙ. Of course, other types of long-range communication interfaces may also be used in accordance with the present invention.

### Computing Devices

Referring back to FIG. 1, container vaults 104₁-104ₙ may transmit notifications to the computing devices 106₁-106ₙ of the users who are authorized to retrieve medication packages from container vaults 104₁-104ₙ (e.g., the intended recipient and/or individuals on the collector list for each medication package). Examples of computing devices 106₁-106ₙ include a smartphone, a personal computing tablet, a smart watch, a personal computer, a laptop computer, or any other computing device that is capable of communication with central server system 102 via network 110. The notifications transmitted from container vaults 104₁-104ₙ to computing devices 106₁-106ₙ will be described below in connection with FIGS. 10A and 10B.

### Servers of Healthcare Providers

Referring still to FIG. 1, central server 102 may transmit notifications to servers 108₁-108ₙ of the healthcare providers located in the communities in which container vaults 104₁-104ₙ are located. In some embodiments, each of servers 108₁-108ₙ broadcasts the notifications to clinicians associated with the healthcare provider. Of course, central server 102 could also transmit the notifications directly to the clinicians. The notifications transmitted from central server 102 to servers 108₁-108ₙ will be described below in connection with FIG. 11.

### Methods/Software Functionality

The description of container vault system 100 provided above references various methods (i.e., software functionality) that are performed by central server 102 and container vaults 104₁-104ₙ. These methods will now be described with reference to FIG. 9 (order management process performed by central server 102), FIGS. 10A-10B (control process performed by the control system of container vaults 104₁-104ₙ), and FIG. 11 (container group assessment process performed by central server 102).

### Order Management Process (Central Server)

Referring to FIG. 9, an order management process performed by central server 102 in accordance with one embodiment of the present invention is described with reference to steps 900-912.

In step 900, central server 102 receives an order for a prescription medication. In this embodiment, a consumer orders and requests delivery of the medication using a conventional online interface and, as part of this process, selects a convenient vault location for pickup of the medication. The consumer may also identify one or more users who are authorized to retrieve the medication package on behalf of the consumer. These users are referred to as “collectors” and are added to a list of collectors. It can be appreciated that certain package data is captured during this step, such as the destination container vault, the physical location of the container vault, the intended recipient of the medication package (i.e., the consumer), and the list of collectors.

A pharmacist who is tasked with filling the prescription then places the prescribed medication in a container, which is then placed in a package for transport to the selected vault location. The pharmacist may also use a software application or online interface to provide information associated with the medication package. It can be appreciated that certain package data is captured during this step, such as the desired environmental properties for storage of the medication package (e.g., temperature level, humidity level, light level, pressure level, or ventilation level), the sender of the medication package (i.e., the pharmaceutical fulfillment system), the medication package value, and the list of medication package category tags. The list of transporters for the medication package may also be provided by the sender, or may alternatively be provided directly by the transporters tasked with delivering the medication package to the selected vault location.

In step 902, central server 102 identifies an available vault compartment at the selected vault location and assigns the GUID of the vault compartment to the medication package. In step 904, central server 102 generates package data for the medication package and stores the package data in association with the assigned GUID within medication package database 208. In step 906, central server 102 transmits the package data along with the assigned GUID to the appropriate container vault. The steps performed by the container vault will be described below in connection with FIGS. 10A and 10B.

In step 908, central server 102 receives periodic messages from the control system of the container vault with information relating to the contents of the vault, including updated package data for the medication package stored in the vault compartment having the assigned GUID. Central server then updates the package data stored in medication package database 208 with the updated information. In step 910, central server 102 determines if the medication package has been retrieved from the container vault. If not, then the process returns to step 908. However, if the medication package has been retrieved from the container vault, then the GUID is marked as unassigned in step 912 so that the GUID is available for assignment to another medication package.

Of course, it should be understood that steps 900-912 are performed for each of different medication packages transported to different container vaults in order to fulfill different orders for prescription medications.

### Control Process (Control System of Container Vault)

Referring to FIGS. 10A and 10B, a control process performed by the control system of one of container vaults 104₁-104ₙ (e.g. container vault 104 described above in connection with FIGS. 3-8) in accordance with one embodiment of the present invention is described with reference to steps 1000-1022.

In step 1000, the control system of container vault 104 receives package data for a medication package along with an assigned GUID from central server 102. The package data includes configuration data that identifies at least one environmental property setting to be used for storage of the medication package within container 500, as well as user access data that identifies at least one authorized user permitted to retrieve the medication package from the container vault. Of course, it should be understood that other information is also included in the package data, as described above. The GUID corresponds to the vault compartment of container vault 104 in which the medication package is to be placed.

In step 1002, the control system of container vault 104 primes the container 500 within the vault compartment associated with the GUID. Specifically, the environmental conditions within container 500 are adjusted to achieve the environmental property settings included in the package data. As described above, the control system transmits the environmental property settings received from central server 102 to electronic circuit 610 of flooring section 600 of container 500, which controls the adjustment of the environmental conditions within container 500 to achieve the environmental property settings. In some embodiments, the control system automatically transmits the environmental property settings to electronic circuit 610 upon receipt of the package data from central server 102 (or within a predetermined amount of time thereafter). In other embodiments, the process is initiated by a technician responsible for preparing container 500 for receipt of the medication package, such as by entering commands via the user interface of touchscreen display 306 of container vault 104.

In step 1004, the control system of container vault 104 determines if the medication package has been placed in container 500 within the vault compartment associated with the GUID. Preferably, upon receipt of the medication package, a technician checks the medication package for quality and accuracy and then places the medication in container 500 of the appropriate vault compartment. In some embodiments, the control system determines that the medication package has been placed in container 500 if the technician has logged a vault entry time via the user interface of touchscreen display 306 of container vault 104. In other embodiments, the control system determines that the medication package has been placed in container 500 upon detection of the opening of the door of the vault compartment and/or detection of the weight of the medication package within container 500 (e.g., using a digital scale), in which case the vault entry time is automatically populated. If the medication package has not been placed in container 500, then the process returns to step 1004. However, if the medication package has been placed in container 500, then the status of the medication package is updated in step 1006.

In step 1008, the control system of container vault 104 transmits a notification to the consumer (i.e., the intended recipient) indicating that the medication package is ready for pickup. At this time, the consumer may be given the opportunity to update the list of collectors if desired. A PIN may also be provided within the notification, which can be used by the consumer to provide the appropriate access credentials at the time of pickup. Of course, it should be understood that the control system of container vault 104 could send a message to central server 102 indicating that the medication package is ready for pickup, in which case the notification to the consumer could originate from central server 102.

In step 1010, the control system of container vault 104 determines if a user has requested access to the container vault to retrieve the medication package from container 500. If not, then it is determined in step 1012 whether the amount of time that the medication package has been stored in container 500 is greater than a predetermined threshold. For example, an expiry time may be calculated based on the vault entry time, in which case it is determined whether the current time is past the expiry time. If not, then the process returns to step 1010. However, if the expiry time has passed, then, in step 1014, control system of container vault 104 transmits a notification to the consumer (i.e., the intended recipient) with a reminder that the medication package is ready for pickup. In some embodiments, the reminder notification may include an offer of a monetary discount (if the failure to retrieve the medication is due to financial issues) or may include educational materials (if the failure to retrieve the medication stems from ignorance on the benefits of the medication). These temporal settings would also enable insights into the expiration date of the medication. Again, it should be understood that this reminder notification could also originate from central server 102, as noted above.

In step 1016, if the user has requested access to the container vault to retrieve the medication package from container 500, then the control system determines if the access credentials provide by the user are valid. For example, the identity of the user may be determined from the information read by card reader 308 or barcode scanner 310 and compared to the identity of the intended recipient and/or collectors provided in the package data. If the user has input a PIN via the user interface of touchscreen display 306, then the PIN may be compared to the PIN provided to the consumer in the notifications sent in step 1008. If the access credentials are not valid, then access is denied in step 1018 and the process returns to step 1010. However, if the access credentials are valid, then the control system unlocks the door of the vault compartment to enable access to container 500 and the medication package stored therein. Then, in step 1022, the control system logs the time that the medication package is retrieved from the container vault and updates the status of the medication package. This information is also provided to central server 102 so as to update the package data in medication package database 208.

Of course, it should be understood that steps 1000-1022 are performed by the control system of each of the different container vaults in relation to each of the medication packages placed in the container of one of the vault compartments.

### Container Group Assessment Process (Central Server)

Referring to FIG. 11, a container group assessment process performed by central server 102 in accordance with one embodiment of the present invention is described with reference to steps 1100-1108.

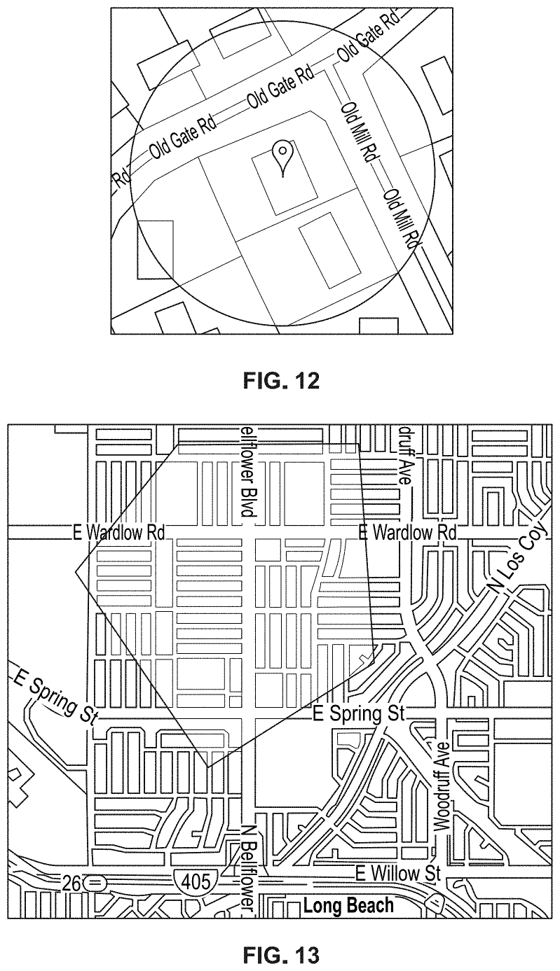

In step 1100, central server 102 creates a geofence (i.e., a virtual fence or perimeter) around the physical location of one of container vaults 104₁-104ₙ (e.g. container vault 104 described above in connection with FIGS. 3-8). In some embodiments, central server 102 obtains the latitude/longitude coordinates of container vault 104 as provided by a GPS unit located within the vault structure. In other embodiments, the latitude/longitude coordinates of container vault 104 are previously stored in memory 202 of central server 102. A circular geofence is then created that is centered around the physical location of container vault 104. In this example, the radius of the circular geofence is set to three miles, i.e., the geofence covers all points located three miles or less from the vault location, although a different radius may be used with other container vaults. An example of a circular geofence is shown in FIG. 12.

In step 1102, central server 102 refines the geofence based on the latitude/longitude coordinates of the home address of each consumer who retrieves a medication package from container vault 104 (either directly or through a designated collector), provided that the home address is located within the circular geofence. Each of these latitude/longitude coordinates are used to form a boundary node, and the boundary nodes are connected to form a polygon geofence. An example of a polygon geofence is shown in FIG. 13.

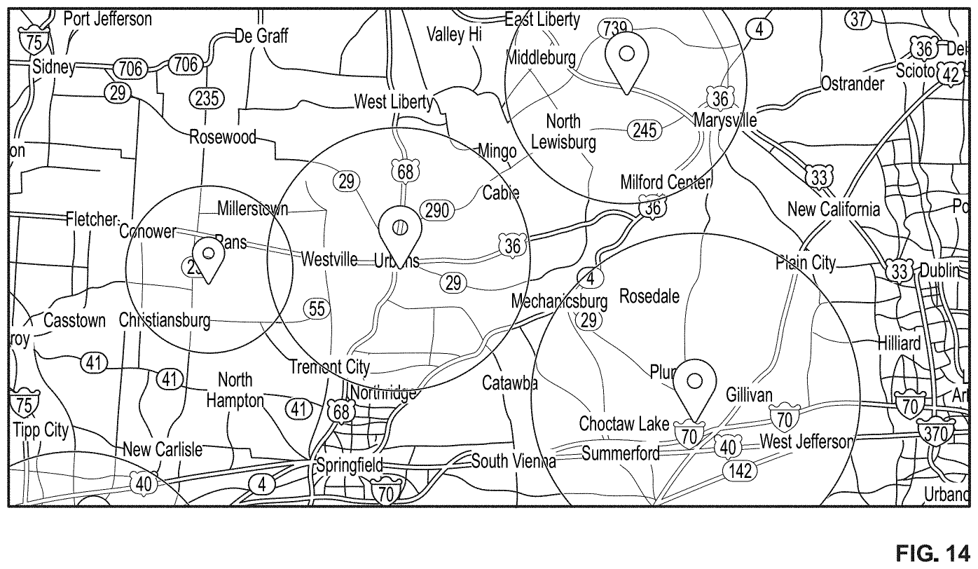

In step 1104, central server 102 defines a container group that includes one or more of container vaults 104₁-104ₙ so as to form a container vault community. An example container group is shown in FIG. 14, which includes four container vaults located in relative close proximity to each other. In this example, each of the container vaults is associated with a circular geofence. Of course, a polygon geofence may alternatively be used in other examples.

In step 1106, central server 102 identifies a group characteristic associated with the container group and stores the group characteristic in container group database 210. The group characteristic may be identified based on an analysis of at least a portion of the package data for the medication packages retrieved from the container vaults within the group as stored in medication package database 208. In some embodiments, the group characteristic comprises a medical condition associated with the group. In other embodiments, the group characteristic comprises a medical supply needed for the group. Thus, central server 102 is able to identify the aggregate afflictions and medical needs of a community.

In step 1108, central server transmits a notification of the group characteristic to the servers 108₁-108ₙ of one or more health care providers who serve patients in proximity to the group. In some embodiments, the notification includes educational materials for a medical condition associated with the group. In other embodiments, the notification includes a medical supply list for the group. It should be understood that the notification may alert local medical facilities of a forthcoming medical condition and provide educational rubrics with the latest in findings on the medical condition as well as insights of medical supplies needed to treat the medical condition.

It should be understood that central server 102 may analyze the group data stored in container group database 210 in order to track a group characteristic within a group or track a group characteristic in relation to one or more additional groups. Trends of “community afflictions” can be tracked as they migrate from one area to another thereby enabling pandemic/flu tracking as the needs from one area spread or migrate entirely. As an example, central server 102 could identify the initial communities dealing with “advanced pneumonia” symptoms (such as those seen with Covid-19) and thereby allow for early quarantine and the acquisition of necessary personal protective equipment (PPE) and respiratory equipment. Central server 102 could also view the progression of a local population's health dynamics such that proactive actions are feasible by local clinicians, hospitals and other health care providers.

Central server 102 may also use the package data within medication package database 208 to provide other services. For example, central server 102 may analyze the medication package category tags in the package data for a particular medication package in combination with the personal health information (PHI) of the consumer to provide recommendations on available services (e.g., healthy catering options or dietary education classes to clients who order blood pressure medication).

Also, central server 102 may use the package data within medication package database 208 to provide both predictive and prescriptive analytical insights. Predictive analytics would leverage the package data to predict future trends, epidemic tracking, chronic conditional tracking, etc. For example, the package data for the medication packages stored in a particular container vault may be analyzed to determine the needs of the immediate area and also infer “community health conditions” for nearby container vaults. This feature may be used in conjunction with a hospital equipment registry to make more precise equipment inventory recommendations.

Prescriptive analytics would answer “what-if” lines of questioning to provide demand forecasting, predictive pricing strategies, and inventory management to enable dynamic supply chains. Dynamic supply chains would enable patient needs to be fulfilled faster and more efficiently, allowing for expansion into general vending kiosks or courier services for medical needs. Stock can be collocated where it is most needed as proven by community metrics and forecasting. In addition, stock can be moved at the forecasted conclusion of an impact zone and migrated to the next anticipated need-based location. This would be in contrast to conventional methods in which a general warehouse blindly ships items based on order status.

### ALTERNATIVE EMBODIMENTS

Various alternative embodiments to system 100 as described above will be apparent to one skilled in the art.

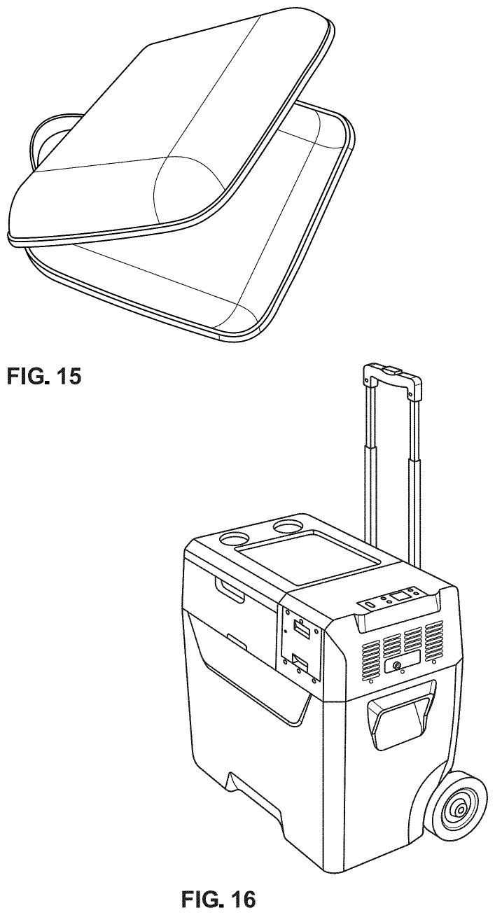

For example, different types of containers may be used that have a configuration different from that of container 500 described above. For example, a brief-case style container, such as the container shown in FIG. 15, may be used to transport a medication package from the pharmaceutical fulfillment system to the vault location and then placed in a vault compartment of the container vault for retrieval by the consumer (or an individual on the collector list). This type of container includes its own rechargeable battery that can be charged at both the vault facility as well as at the consumer's home. The container includes components to control the environmental conditions within the container in order to protect the prescriptive medication stored therein. The consumer may return this container to the vault facility for transport back to the pharmaceutical fulfillment system.

Also, different types of container vaults may be used that have a configuration different from that of container vault 104 described above. For example, the container vault may comprise a single standalone enclosure, such as the chest-like vault shown in FIG. 16, with a container that holds a medication package transported from a pharmaceutical fulfillment system directly to a consumer's home. This type of container vault may be left in the consumer's driveway, and a transporter would have the appropriate access credentials to open the container vault and place the medication package therein. The container also includes components to control the environmental conditions within the container in order to protect the prescriptive medication stored therein. The environmental settings may be automatically set such that the desired temperature, pressure, etc. is achieved prior to arrival of the delivery truck. The container vault also provides secure access similar to the methods described above in connection with container vault 104 such that only trusted individuals are allowed access to its contents.

In addition, while container vault 104 described above includes features that provide secure access to the container and also control the environmental conditions within the container, other container vaults may be used that provide only one of these features (i.e., secure access or controlled environmental conditions, but not both). Further, while container vault 104 described above is used to store medication packages for retrieval by consumers, other types of products may also be placed in the secure containers, such as medical supplies and/or over-the-counter (OTC) generic medications for a wide range of ailments. Moreover, because the container vault can be located in any third party facility, it may provide a convenient option for consumers who do not have easy access to a designated pharmacy.

### General Information

The description set forth above provides several exemplary embodiments of the inventive subject matter. Although each exemplary embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

The use of any and all examples or exemplary language (e.g., “such as” or “for example”) provided with respect to certain embodiments is intended merely to better describe the invention and does not pose a limitation on the scope of the invention. No language in the description should be construed as indicating any non-claimed element essential to the practice of the invention.

The use of the terms “comprises,” “comprising,” or any other variation thereof, are intended to cover a non-exclusive inclusion, such that a system or method that comprises a list of elements does not include only those elements, but may include other elements not expressly listed or inherent to such system or method.

Finally, while the present invention has been described and illustrated hereinabove with reference to various exemplary embodiments, it should be understood that various modifications could be made to these embodiments without departing from the scope of the invention. Therefore, the present invention is not to be limited to the specific systems or methods of the exemplary embodiments, except insofar as such limitations are included in the following claims.

What is claimed and desired to be secured by Letters Patent is as follows:

1. A container vault, comprising:
at least one container configured to hold a medication package, wherein the container includes at least one component configured to change an environmental property within the container; and
a control system configured to:
store package data associated with the medication package, wherein the package data includes (i) configuration data that identifies at least one environmental property setting to be used for storage of the medication package and (ii) user access data that identifies at least one authorized user permitted to retrieve the medication package;
control the component so that the environmental property within the container is adjusted to achieve the environmental property setting; and
control access to the container so that the authorized user is permitted to retrieve the medication package from the container.

2. The container vault of claim 1, wherein the environmental property comprises one of a temperature level within the container, a humidity level within the container, a light level within the container, a pressure level within the container, and a ventilation level within the container.

3. The container vault of claim 1, wherein the control system is configured to receive the package data from a remote server.

4. The container vault of claim 3, wherein the control system is configured to transmit a message to the remote server when the medication package is placed in the container for retrieval by the authorized user.

5. The container vault of claim 3, wherein the control system is configured to transmit a message to the remote server when the authorized user has accessed the container to retrieve the medication package.

6. The container vault of claim 1, wherein the container includes a power source configured to provide power to the component.

7. The container vault of claim 1, further comprising a vault housing configured to house the container in a locked vault, and wherein the control system is configured to control access to the container by (i) obtaining access credentials, (ii) determining if the access credentials are associated with the authorized user, and (iii) if the determination is affirmative, unlocking the vault to enable access to the container.

8. The container vault of claim 7, wherein the control system includes at least one input device configured to obtain the access credentials, wherein the input device comprises one of a card reader, a barcode scanner, a touchscreen display, a keypad, and a biometric reader.

9. A container vault system, comprising:
a server configured to store package data for each of a plurality of medication packages; and
a plurality of container vaults each of which is configured to communicate with the server via a communication network, wherein each of the container vaults comprises:
at least one container configured to hold a medication package; and
a control system configured to (i) receive the package data for the medication package from the server, wherein the package data identifies at least one authorized user permitted to retrieve the medication package and (ii) control access to the container so that the authorized user identified in the package data is permitted to retrieve the medication package from the container.

10. The container vault system of claim 9, wherein the package data for the medication package identifies at least one environmental property setting to be used for storage of the medication package, wherein the container includes at least one component configured to change an environmental property within the container, and wherein the control system is configured to control the component so that the environmental property within the container is adjusted to achieve the environmental property setting identified in the package data.

11. The container vault system of claim 10, wherein the environmental property comprises one of a temperature level within the container, a humidity level within the container, a light level within the container, a pressure level within the container, and a ventilation level within the container.

12. The container vault system of claim 10, wherein the container includes a power source configured to provide power to the component.

13. The container vault system of claim 9, wherein the control system is configured to transmit a message to the server when the medication package is placed in the container for retrieval by the authorized user.

14. The container vault system of claim 13, wherein the server is configured to transmit a notification to a computing device of the authorized user upon receipt of the message.

15. The container vault system of claim 13, wherein the server is configured to transmit a notification to a computing device of the authorized user if the medication package is not retrieved from the container within a predetermined time period.

16. The container vault system of claim 9, wherein the control system is configured to transmit a message to the server when the authorized user has accessed the container to retrieve the medication package.

17. The container vault system of claim 9, wherein the server is configured to (i) define a group containing one or more of the container vaults and (ii) analyze at least a portion of the package data for the medication packages retrieved from the one or more container vaults within the group to identify a group characteristic.

18. The container vault system of claim 17, wherein the group characteristic comprises a medical condition associated with the group or a medical supply needed for the group.

19. The container vault system of claim 17, wherein the server is configured to track the group characteristic within the group or in relation to one or more additional groups.

20. The container vault system of claim 17, wherein the server is configured to transmit a notification of the group characteristic to one or more computing devices of one or more health care providers.

21. The container vault system of claim 20, wherein the notification of the group characteristic includes educational materials for a medical condition associated with the group or a medical supply list for the group.

22. The container vault system of claim 9, wherein the container vault further comprises a vault housing configured to house the container in a locked vault, and wherein the control system is configured to control access to the container by (i) obtaining access credentials, (ii) determining if the access credentials are associated with the authorized user, and (iii) if the determination is affirmative, unlocking the vault to enable access to the container.

23. The container vault system of claim 22, wherein the control system includes at least one input device configured to obtain the access credentials, wherein the input device comprises one of a card reader, a barcode scanner, a touchscreen display, a keypad, and a biometric reader.
